(19) United States
(12) Patent Application Publication
(10) Pub. No.: US 2006/0166969 A1
(43) Pub. Date: Jul. 27, 2006

(54) AMPA antagonists for the treatment of dizziness, including vertigo and Meniere's disorder

(76) Inventors: Lechoslaw A. Turski, Weesp (NL); Bernard J. Van Vliet, Weesp (NL); Rene Remie, Weesp (NL); Johannes A.M. Van Der Heijden, Weesp (NL); Martinus Th. M. Tulp, Weesp (NL)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/331,026

(22) Filed: Jan. 13, 2006

Related U.S. Application Data
(60) Provisional application No. 60/643,598, filed on Jan. 14, 2005.

(51) Int. Cl. A61K 31/551

(52) U.S. Cl. 514/219

(57) ABSTRACT

The disclosure relates to the use of 8,9-dimethyl-5-phenyl-11H-1,3-dioxolo[4,5-h]-imidazo[1,2-c][2,3]benzodiazepine: 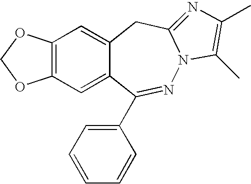 prodrugs, tautomers and N-oxides thereof, as well as pharmacologically acceptable salts, hydrates and solvates of the foregoing, for the preparation of a pharmaceutical composition for the treatment, amelioration or prevention of dizziness, including vertigo and Menière's disorder.

This application claims benefit of U.S. Provisional Application No. 60/643,598, filed Jan. 14, 2005, the content of which is incorporated herein by reference.

One aspect of the present disclosure relates to a novel use of 8,9-dimethyl-5-phenyl-11H-1,3-dioxolo[4,5-h]-imidazo[1,2-c][2,3]benzodiazepine for the preparation of medicaments for treatment of dizziness, including vertigo and Menière's disorder.

Another aspect of the present disclosure relates to the use of the compound disclosed herein for the manufacture of a medicament giving a beneficial effect. A beneficial effect is disclosed herein or apparent to a person skilled in the art from the specification and general knowledge in the art.

The present disclosure also relates to the use of the compound of the present disclosure for the manufacture of a medicament for treating or preventing a disease or condition. Furthermore, the present disclosure relates to a new use for the treatment of a disease or condition disclosed herein or apparent to a person skilled in the art from the specification and general knowledge in the art. In at least one embodiment of the present disclosure the specific compound disclosed herein is used for the manufacture of a medicament.

As a complaint rather than a disease, the term “dizziness” appropriately includes almost any sensation that a patient chooses to call by that name. By usage the term is applied to a number of uncommon sensations that are not part of daily experience. It is the thread of unfamiliar spatial disorientation that appears to bind these complaints together. Experienced practitioners are able to distinguish several distinct types of dizziness. When a definite rotational sensation is present, in other words when the patient feels that either he or the environment is spinning, the diagnosis is “type I dizziness”, more generally known as “vertigo”. A sensation of impending faint or loss of consciousness is termed “type II dizziness”, while “type III dizziness” is reserved for disequilibrium. Other forms of dizziness, and also those that cannot be identified with certainty as not being vertigo, faintness or disequilibrium are collected under the term “type IV dizziness”.

Menière's disorder, which is also known as Menière's disease or Menière's syndrome, is widely considered to be one of the most frequent causes of dizziness, but it actually accounts for only about 5% of all dizziness and for 10 to 15% of vertigo. It usually occurs in adults and consists of recurring bouts of vertigo associated with hearing loss and tinnitus which may precede or follow the first bout of vertigo.

Current pharmacotherapeutic treatment of dizziness, vertigo or Menie re's disorder consists of a number of different drugs. What they have in common is that for none of them is there clear insight whether or not their main mechanism of action contributes to their purported anti-vertigo effects.

Betahistine is a weak histamine-H₁ agonist and somewhat more potent histamine-H₃ antagonist that stimulates the synthesis and release of histamine. Clinical improvement of the symptoms of Menière's disorder in most cases is apparent only after weeks of treatment, while optimal effects are reached after months.

Cinnarizine and its fluorinated analog flunarizine are antihistaminics with modest anticholinergic effects as well as calcium-antagonistic effects.

Piracetam is sometimes referred to as chemically related to the endogenous neurotransmitter GABA, but its mechanism of action is unclear.

The mechanism of action of gabapentin is complex; it is not a GABA receptor agonist, yet elevates GABA levels in the brain, by a mechanism most likely associated with voltage-sensitive Ca²⁺ channels. Gabapentin also has an activity as AMPA antagonist (Chizh et al., Naunyn-Schmiedeberg's Arch. Pharmacol (2000), 362, 197-200).

Sulpiride, an atypical antipsychotic, is best known for its dopaminolytic effect.

The mechanism of action of acetyl-d,/-leucine is unknown.

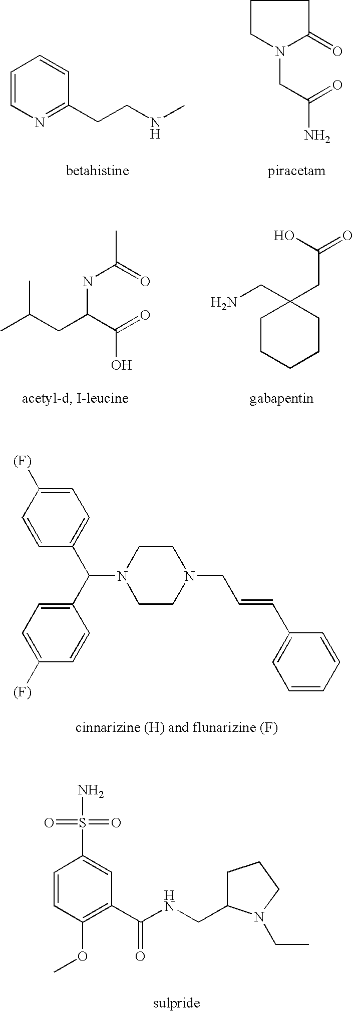

As stated above, for none of the drugs described above is it clear whether or not their mechanism of action contributes to their purported anti-vertigo effects. Even worse, for none of the specific anti-vertigo drugs has clinical efficacy over placebo been unambiguously demonstrated.

What is clear, however, is that the drugs in question have side-effects. For betahistine these are not severe, and include some gastrointestinal complaints and an odd headache. Also, piracetam produces gastrointestinal side effects, but more serious are nervousness, depression and anxiety. Cinnarizine and flunarizine give rise to depression, sedation and Parkinsonism, the latter being also the most adverse of the side effects of sulpiride, also known for its extrapyramidal side effects. Gabapentin may also cause sedation and nausea.

Another aspect of the present disclosure is to develop a therapy for dizziness, including vertigo and Menière's disorder, alternative to the currently available therapies, i.e., a compound with an improved efficacy and better tolerated side effect profile.

Surprisingly, 8,9-dimethyl-5-phenyl-11H-1,3-dioxolo[4,5-h]imidazo[1,2-c][2,3]benzo-diazepine: 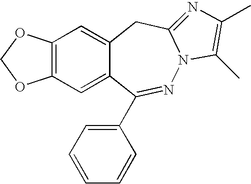
has been found active in animal models for dizziness, including vertigo and Menière's disorder.

One aspect of the present disclosure relates to the use of this compound, tautomers and N-oxides thereof, as well as pharmacologically acceptable salts, hydrates and solvates of this compound and its tautomers and N-oxides, for the preparation of a pharmaceutical composition for the treatment, amelioration or prevention of dizziness, including vertigo and Menière's disorder. The synthesis of the compound has been disclosed in WO 97/28163 and WO 02/12247.

Prodrugs of the compound mentioned above are within the scope of the present disclosure. Prodrugs are therapeutic agents which are inactive per se but are transformed into one or more active metabolites. Prodrugs are bioreversible derivatives of drug molecules used to overcome some barriers to the utility of the parent drug molecule. These barriers include, but are not limited to, solubility, permeability, stability, presystemic metabolism and targeting limitations (Medicinal Chemistry: Principles and Practice, 1994, ISBN 0-85186-494-5, Ed.: F. D. King, p. 215; J. Stella, “Prodrugs as therapeutics”, Expert Opin. Ther. Patents, 14(3), 277-280, 2004; P. Ettmayer et al., “Lessons learned from marketed and investigational prodrugs”, J. Med. Chem., 47, 2393-2404, 2004). Pro-drugs, i.e., compounds which, when administered to humans by any known route, are metabolised to 8,9-dimethyl-5-phenyl-11H-1,3-dioxolo[4,5-h]imidazo[1,2-c][2,3]benzo-diazepine, are included within the present disclosure.

N-oxides of the compound mentioned above are within the scope of the present disclosure. Tertiary amines may or may not give rise to N-oxide metabolites. The extent to which N-oxidation takes place varies from trace amounts to a near quantitative conversion. N-oxides may be more active than their corresponding tertiary amines or less active. While N-oxides are easily reduced to their corresponding tertiary amines by chemical means, in the human body this happens to varying degrees. Some N-oxides undergo nearly quantitative reductive conversion to the corresponding tertiary amines, in other cases the conversion is a mere trace reaction or even completely absent. (M. H. Bickel: “The pharmacology and Biochemistry of N-oxides”, Pharmacological Reviews, 21(4), 325- 355, 1969).

Pharmaceutically acceptable salts may be obtained using standard procedures well known in the art, for example by mixing the compound of the present disclosure with a suitable acid, such as, for instance, an inorganic acid or an organic acid.

8,9-Dimethyl-5-phenyl-11H-1,3-dioxolo[4,5-h]imidazo[1,2-c][2,3]benzo-diazepine, as well as the pharmacologically acceptable salts thereof, has AMPA receptor antagonistic activity. It is useful in the treatment, amelioration or prevention of dizziness, including vertigo and Menière's disorder.

### PHARMACOLOGICAL METHODS

Animal models for dizziness, vertigo and Menière's disorder include, but are not limited

Electronystagmography in rats (see Fischer, A. J. et al., “Electronystagmography in the laboratory rat”, Acta Otolaryngol., 88, 412-419,1979).

Gentamycin induced nystagmus in guinea pigs (see Okuda, T. et al., “Inner ear changes with intracochlear gentamicin administration in Guinea pigs”, Laryngoscope, 114, 694-697, 2004).

Unilateral peripheral vestibular deafferentation in guinea pigs (see Dutia, M. B. et al., “The opioid receptor antagonist, naloxone, enhances ocular motor compensation in guinea pig following peripheral vestibular deafferentation”, Exp. Neurol., 141,141-144,1996), or rats (see Guilding, C. et al., “11-Beta-hydroxysteroid dehydrogenase type 1 activity in medial vestibular nucleus and cerebellum after unilateral vestibular deafferentation in the rat”, Stress, 7(2),127-130, 2004).

Unilateral labyrinthectomy in rats (see Johnston, A. R. et al., “Differential regulation of GABA(A) and GABA(B) receptors during vestibular compensation”, Neuroreport.;12(3), 597-600, 2001; and Inoue, S. et al., “Glutamate release in the rat medial vestibular nucleus following unilateral labyrinthectomy using in vivo microdialysis”, Brain Res., 991(1-2), 78-83, 2003).

Unilateral vestibular neurectomized cats (see Tighilet, B. et al, “Betahistine dihydrochloride treatment facilitates vestibular compensation in the cat”, J. Vestib. Res., 5(1), 53-66, 1995; Tighilet, B. et al, “Gamma amino butyric acid (GABA) immunoreactivity in the vestibular nuclei of normal and unilateral vestibular neurectomized cats”, Eur. J. Neurosci., 3(12):2255-2267, 2001).

Excitation of rat medial vestibular nucleus neurons in vitro (see Dutia, M. B., “Betahistine, vestibular function and compensation: in vitro studies of vestibular function and plasticity”, Acta Otolaryngol Suppl. 544:11-14, 2000), or in vivo (see Yamanaka, T. et al., “Rapid compensatory changes in GABA receptor efficacy in rat vestibular neurones after unilateral labyrinthectomy”, J. Physiol., 523, 413-424, 2000).

### Pharmaceutical Preparations

8,9-Dimethyl-5-phenyl-11H-1,3-dioxolo[4,5-h]imidazo[1,2-c][2,3]benzo-diazepine can be brought into forms suitable for administration by means of usual processes using auxiliary substances such as liquid or solid carrier material. The pharmaceutical compositions of the present disclosure may be administered enterally, orally, parenterally (intramuscularly or intravenously), rectally or locally (topically). They can be administered in the form of solutions, powders, tablets, capsules (including microcapsules), ointments (creams or gel) or suppositories. Suitable excipients for such formulations are the pharmaceutically customary liquid or solid fillers and extenders, solvents, emulsifiers, lubricants, flavorings, colorings and/or buffer substances. Frequently used auxiliary substances which may be mentioned are magnesium carbonate, titanium dioxide, lactose, mannitol and other sugars or sugar alcohols, talc, lactoprotein, gelatin, starch, cellulose and its derivatives, animal and vegetable oils such as fish liver oil, sunflower, groundnut or sesame oil, polyethylene glycol and solvents such as, for example, sterile water and mono- or polyhydric alcohols such as glycerol.

Types of pharmaceutical compositions that may be used include, but are not limited to, tablets, chewable tablets, capsules, solutions, parenteral solutions, suppositories, suspensions, and other types disclosed herein or apparent to a person skilled in the art from the specification and general knowledge in the art.

In at least one embodiment of the present disclosure, a pharmaceutical pack or kit is provided comprising at least one container filled with at least one of the ingredients of a pharmaceutical composition of the present disclosure. Associated with the at least one container can be various written materials such as instructions for use, or a notice in the form prescribed by a governmental agency regulating the manufacture, use or sale of pharmaceuticals products, which notice reflects approval by the agency of manufacture, use, or sale for human or veterinary administration.

### Dose

The affinity of 8,9-dimethyl-5-phenyl-11H-1,3-dioxolo[4,5-h]imidazo[1,2-c][2,3]benzo-diazepine for AMPA receptors has been published. From the binding affinity measured for a given compound, one can estimate a theoretical lowest effective dose. At a concentration of the compound equal to twice the measured Kᵢ-value, 100% of the AMPA receptors likely will be occupied by the compound. Converting that concentration to mg of compound per kg of patient yields a theoretical lowest effective dose, assuming ideal bioavailability. Pharmacokinetic, pharmacodynamic, and other considerations may alter the dose actually administered to a higher or lower value. The dosage expediently administered is 0.001-1000 mg/kg, such as, for example, 0.1-100 mg/kg of a patient's bodyweight.

### Treatment

The term “treatment” as used herein refers to any treatment of a mammalian, such as, for example, treatment of a human condition or disease, and includes: (1) preventing the disease or condition from occurring in a subject which may be predisposed to the disease but has not yet been diagnosed as having it; (2) inhibiting the disease or condition, i.e., arresting its development; (3) relieving the disease or condition, i.e., causing regression of the condition; and (4) relieving the conditions caused by the disease, i.e., stopping the symptoms of the disease.

### Formulations for Animal Studies

For oral (p.o.) administration: to the desired quantity (0.5-5 mg) of the solid 8,9-dimethyl-5-phenyl-11H-1,3-dioxolo[4,5-h]imidazo[1,2-c][2,3]benzodiazepine in a glass tube, some glass beads were added and the solid was milled by vortexing for 2 minutes. After addition of 1 ml of a solution of 1% methylcellulose in water and 2% (v/v) of Poloxamer 188 (Lutrol F68), the compound was suspended by vortexing for 10 minutes. The pH was adjusted to 7 with a few drops of aqueous NaOH (0.1N). Remaining particles in the suspension were further suspended by using an ultrasonic bath.

For intraperitoneal (i.p.) administration: to the desired quantity (0.5-15 mg) of the solid 8,9-dimethyl-5-phenyl-11H-1,3-dioxolo[4,5-h]imidazo[1,2-c][2,3]benzodiazepine in a glass tube, some glass beads were added and the solid was milled by vortexing for 2 minutes. After addition of 1 ml of a solution of 1% methylcellulose and 5% mannitol in water, the compound was suspended by vortexing for 10 minutes. Finally the pH was adjusted to 7.

1. A method of preparing a pharmaceutical composition comprising combining at least one compound chosen from 8,9-dimethyl-5-phenyl-11H-1,3-dioxolo[4,5-h]imidazo[1,2-c][2,3]benzo-diazepine, 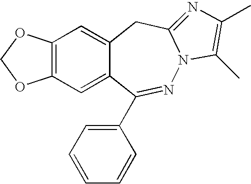
a prodrug thereof, a tautomer thereof, an N-oxide thereof, or a pharmacologically acceptable salt thereof, or at least one hydrate or solvate of any of the foregoing or a mixture of any two or more of the foregoing with at least one pharmaceutically acceptable carrier, at least one pharmaceutically acceptable auxiliary substance, or a combination thereof, wherein the at least one compound is present in an amount effective for the treatment, amelioration or prevention of dizziness.

2. The method according to claim 1, wherein the 8,9-dimethyl-5-phenyl-11H-1,3-dioxolo[4,5-h]imidazo[1,2-c][2,3]benzo-diazepine, a prodrug thereof, a tautomer thereof, an N-oxide thereof, or a salt thereof, or a hydrate or solvate of any of the foregoing is present in an amount effective for the treatment, amelioration or prevention of vertigo.

3. The method according to claim 1, wherein the 8,9-dimethyl-5-phenyl-11H-1,3-dioxolo[4,5-h]imidazo[1,2-c][2,3]benzo-diazepine, a prodrug thereof, a tautomer thereof, an N-oxide thereof, or a salt thereof, or a hydrate or solvate of any of the foregoing is present in an amount effective for the treatment, amelioration or prevention of Menière's disorder.
